(19) United States
(12) Patent Application Publication
(10) Pub. No.: US 2026/0285820 A1
(43) Pub. Date: Sep. 24, 2026

(54) PROCESS FOR PURIFYING 2-PHENYL-4-(3-CARBOALKOXYPROPIONYL)-1,3-OXAZOLIN-5-ONE AND NEW CRYSTALLINE FORM B OF 2-PHENYL-4-(3-CARBOMETHOXYPROPIONYL)-1,3-OXAZOLIN-5-ONE

(71) Applicant: HERAEUS PRECIOUS METALS GMBH & CO. KG, Hanau (DE)

(72) Inventors: Tero KUNNARI, Hanau (DE); Jenin GREIN, Hanau (DE)

(21) Appl. No.: 19/151,471

(22) PCT Filed: Jan. 23, 2024
(86) PCT No.: PCT/EP2024/051507

§ 371 Date: Jul. 28, 2025

(30) Foreign Application Priority Data
Feb. 2, 2023 (EP) 23154635.9

(51) Int. Cl. C07D 263/42; C07C 227/18

(52) U.S. Cl. CPC C07D 263/42; C07C 227/18

(57) ABSTRACT

The present invention relates to a process for purifying 2-phenyl-4-(3-carboalkoxypropionyl)-1,3-oxazolin-5-one, which is formed by reacting N-benzoylglycine with succinic acid monoalkyl ester chloride in 4-methylpyridine and obtainable in the form of red-colored crystals after neutralization with an excess of hydrochloric acid, characterized in that the red-colored crystals are suspended in an organic solvent or a mixture of a plurality of organic solvents until decolorization, the organic solvent or solvents being selected from the group consisting of organic solvents having an elution strength E₀ in the range of from 0.40 to 0.55, and the organic solvent or solvents having a water content in the range of from 1 to 10 vol. %.

The present invention relates to a process for purifying 2-phenyl-4-(3-carboalkoxypropionyl)-1,3-oxazolin-5-one, which is formed by reacting N-benzoylglycine with succinic acid monoalkyl ester chloride in 4-methylpyridine and obtainable in the form of red-colored crystals after neutralization with an excess of hydrochloric acid. The invention also relates to 2-phenyl-4-(3-carboalkoxypropionyl)-1,3-oxazolin-5-one purified by the purification process according to the invention, and to a new crystalline form B of 2-phenyl-4-(3-carbomethoxypropionyl)-1,3-oxazolin-5-one. The invention further relates to the use of 2-phenyl-4-(3-carboalkoxypropionyl)-1,3-oxazolin-5-one purified by the process according to the invention for the preparation of 5-aminolevulinic acid hydrochloride or other salts of 5-aminolevulinic acid.

DE 22 08 800 (A1) discloses the preparation of 5-aminolevulinic acid hydrochloride by hydrolysis of 2-phenyl-4-(3-carbomethoxypropionyl)-1,3-oxazolin-5-one in dilute hydrochloric acid. DE 22 08 800 (A1) also discloses the preparation of the starting product 2-phenyl-4-(3-carbomethoxypropionyl)-1,3-oxazolin-5-one as red-colored crystals by reacting N-benzoylglycine with succinic acid monomethyl ester chloride in 4-methylpyridine and overneutralization with hydrochloric acid. As has now become apparent during the applicant's development work, it is not possible to eliminate the red coloration of the crystals using the washing process described in Example 1 of DE 22 08 800 (A1).

The object of the invention was to provide 2-phenyl-4-(3-carboalkoxypropionyl)-1,3-oxazolin-5-one with a higher purity than that of the red-colored crystalline material prepared analogously to DE 22 08 800 (A1), in particular to provide 2-phenyl-4-(3-carbomethoxypropionyl)-1,3-oxazolin-5-one with a higher purity than that of the red-colored crystalline material obtainable in accordance with Example 1 from DE 22 08 800 (A1).

As can be seen from the following description, the applicant was surprised to find that the impurities causing the red coloration in 2-phenyl-4-(3-carboalkoxypropionyl)-1,3-oxazolin-5-one, especially 2-phenyl-4-(3-carbomethoxypropionyl)-1,3-oxazolin-5-one, which can be prepared analogously or in accordance with DE 22 08 800 (A1), can be removed in a surprisingly simple and effective manner by suspending the respective red-colored crystals for a sufficiently long time in a moist organic solvent of suitable polarity, more precisely in an organic solvent of suitable polarity and with a specifically adjusted water content. However, as the applicant was also surprised to discover, during said suspension not only the impurities can be removed and thus 2-phenyl-4-(3-carboalkoxypropionyl)-1,3-oxazolin-5-one recovered, which is more suitable for the production of 5-aminolevulinic acid hydrochloride or other salts of 5-aminolevulinic acid because it is purer; rather, for example, the known crystalline form A of said red-colored 2-phenyl-4-(3-carbomethoxypropionyl)-1,3-oxazolin-5-one obtainable in accordance with Example 1 of DE 22 08 800 (A1) can be converted into a new crystalline form B during said suspension.

The invention therefore relates to a process for purifying 2-phenyl-4-(3-carboalkoxypropionyl)-1,3-oxazolin-5-one, which is formed by reacting N-benzoylglycine with succinic acid monoalkyl ester chloride in 4-methylpyridine and obtainable in the form of red-colored crystals after neutralization with an excess of hydrochloric acid, characterized in that the red-colored crystals are suspended in an organic solvent or a mixture of a plurality of organic solvents until decolorization, the organic solvent or solvents being selected from the group consisting of organic solvents having an elution strength E⁰ in the range of from 0.40 to 0.55, and the organic solvent or solvents having a water content in the range of from 1 to 10 vol. %.

2-phenyl-4-(3-carboalkoxypropionyl)-1,3-oxazolin-5-one, preferably 2-phenyl-4-(3-carbo-C1-C4-alkoxypropionyl)-1,3-oxazolin-5-one and particularly preferably 2-phenyl-4-(3-carbomethoxypropionyl)-1,3-oxazolin-5-one, which is to be purified by means of the process according to the invention and is present in the form of red-colored crystals, can, as already mentioned, be prepared analogously or in accordance with DE 22 08 800 (A1), in particular analogously or in accordance with Example 1 therein. In this case, the purity of the relevant 2-phenyl-4-(3-carboalkoxypropionyl)-1,3-oxazolin-5-one, which is present in the form of red-colored crystals and determined by means of HPLC is in each case for example in the range of from 90 to 95 HPLC area %, i.e., generally not higher than 95 HPLC area %; in this case, the HPLC area % specification refers to non-volatile material, i.e., to dried material freed from organic solvent or, in the case of material still comprising organic solvent residue, to the proportion of material without organic solvent residue. In other words, 2-phenyl-4-(3-carboalkoxypropionyl)-1,3-oxazolin-5-one (hereinafter also referred to as “original 2-phenyl-4-(3-carboalkoxypropionyl)-1,3-oxazolin-5-one”) to be purified by means of the process according to the invention does not have to be completely dried; it may comprise a residue of organic solvent which is not included in the purity consideration. Such a proportion of organic solvent can in particular originate from a washing step downstream of the synthesis of 2-phenyl-4-(3-carboalkoxypropionyl)-1,3-oxazolin-5-one and, for example, remain after suction filtration in the washed but still not completely purified red-colored crystals; such a residual proportion of organic solvent can, for example, be in the range of up to 10 grams per 100 grams of solvent-moist red-colored crystals.

The aforementioned purity determination by means of HPLC can be carried out, for example, using the following parameters: column (250×4.6 mm, RP-18, 5 μm), injection 5 μL, flow 1 mL/min, column temperature 25° C., UV detection at 254 nm, mobile phase A=0.1% trifluoroacetic acid (TFA) in water, mobile phase B=0.1% TFA in acetonitrile, gradient from 90% A to 10% A within 15 min.

It is essential that the organic solvent used as a liquid suspension medium in the process according to the invention, or the organic solvents contained as a liquid suspension medium in the used mixture of a plurality of organic solvents are selected from the group consisting of organic solvents with an elution strength E⁰ in the range of from 0.40 to 0.55. In the case of the elution strength E⁰ mentioned here this is an empirical value according to the concept of the elutropic series known to a person skilled in the art [cf. W. Trappe; Biochem. Z. 305, 150 (1940); 306, 316 (1940)]. The elutropic series sorts organic solvents according to their elution effect (elution selectivity) during chromatography, in this case with silica gel as the adsorbent.

Examples of such organic solvents with an elution strength E⁰ in the range of from 0.40 to 0.54 comprise acetone (E⁰=0.43), dioxane (E⁰=0.43), ethyl acetate (E⁰=0.45), tetrahydrofuran (E⁰=0.48), acetonitrile (E⁰=0.50). Preferred examples comprise acetone, acetonitrile and ethyl acetate.

It is also essential that the organic solvent or mixture of a plurality of organic solvents used in the process according to the invention should have a water content in the range of from 1 to 10 vol. %, preferably 2 to 5 vol. %. In this case, the water content is always such that the organic solvent or solvents do not form a two-phase system with the water. In this context, a person skilled in the art also speaks of moist organic solvents in contrast to anhydrous organic solvents, known as dried organic solvents.

The suspension taking place in the process according to the invention does not involve any methodological peculiarities and takes place, for example, in the temperature range of from 10 to 40° C., preferably 15 to 30° C.

The suspension can be carried out, for example, in a ratio of 20 to 100 g of 2-phenyl-4-(3-carboalkoxypropionyl)-1,3-oxazolin-5-one per liter of organic solvent, preferably in a ratio of 40 to 60 g of 2-phenyl-4-(3-carboalkoxypropionyl)-1,3-oxazolin-5-one per liter of organic solvent.

The suspension can be achieved, for example, by stirring and/or shaking.

It is also essential that the suspension is carried out until decolorization, i.e., at least until the red-colored crystals have decolorized; there is no harm if the suspension is carried out for a longer period of time. The duration of the suspension can be for example in the range of from 8 to 24 hours. At the beginning or during the suspension, the liquid phase of the suspension takes on a red color, which becomes weaker over time and finally disappears. The decolorization, i.e., the achievement of decolorization, can be recognized visually by a person skilled in the art, for example by the fact that the crystals have lost their red coloration or have become colorless and that preferably the liquid phase has also lost its red coloration, for example only has a weak yellow coloration; said decolorization corresponds to corresponding HPLC measurements with UV/VIS detection at 500 to 600 nm, i.e., in the range of specific absorption of the impurity or impurities causing the red coloration. In other words, the signals originally detectable in this wavelength range decrease during the visually perceptible decolorization procedure until they disappear.

If the suspension is carried out in the same but anhydrous dried organic solvent (mixture) and under otherwise identical conditions, the red coloration does not disappear. It is assumed that the impurities causing the red coloration are chemically changed during suspension in accordance with the process according to the invention, possibly hydrolyzed to form colorless products soluble in the organic solvent (mixture) used, while 2-phenyl-4-(3-carboalkoxypropionyl)-1,3-oxazolin-5-one remains chemically unchanged and can be recovered as crystals, more precisely as colorless crystals, after suspension has ended.

In order to obtain the purified 2-phenyl-4-(3-carboalkoxypropionyl)-1,3-oxazolin-5-one, conventional solid-liquid separation processes known to a person skilled in the art can be used, such as decanting, filtration, suction filtration, centrifugation. The 2-phenyl-4-(3-carboalkoxypropionyl)-1,3-oxazolin-5-one separated in this way and still moist with solvent can then be freed from the organic solvent residue by drying, for example at 20 to 40° C., if necessary assisted by reduced pressure. The purified 2-phenyl-4-(3-carboalkoxypropionyl)-1,3-oxazolin-5-one is colorless or, in other words, freed from its original red coloration, i.e., it is present as colorless crystals.

2-phenyl-4-(3-carboalkoxypropionyl)-1,3-oxazolin-5-one per se, purified and dried by means of the process according to the invention, is chemically unchanged compared to said red-colored 2-phenyl-4-(3-carboalkoxypropionyl)-1,3-oxazolin-5-one which still has to be purified; it does not comprise any water of crystallization, i.e., it is not a hydrate, as could be demonstrated by means of Karl Fischer titration. Its purity, as determinable by HPLC, is higher than that of the red-colored 2-phenyl-4-(3-carboalkoxypropionyl)-1,3-oxazolin-5-one and is generally in the range of from >95 to 99 HPLC area %.

Unexpectedly, 2-phenyl-4-(3-carbomethoxypropionyl)-1,3-oxazolin-5-one purified and dried by means of the process according to the invention has a modified crystalline form B compared to the original, i.e., still unpurified and red-colored 2-phenyl-4-(3-carbomethoxypropionyl)-1,3-oxazolin-5-one, as can be demonstrated by X-ray powder diffraction (XRPD). Thus, the 2-phenyl-4-(3-carbomethoxypropionyl)-1,3-oxazolin-5-one (crystalline form B) purified according to the invention and present in the form of colorless rod-shaped crystals has a powder X-ray diffraction pattern which differs from the red-colored 2-phenyl-4-(3-carbomethoxypropionyl)-1,3-oxazolin-5-one which still has to be purified and which comprises the following reflections: 7.8, 9.1, 13.3, 16.6, 19.5, 19.7, 20.4, 23.0, 23.6, 23.9, 24.1, 24.5, 25.1, 26.1 and 28.0±0.2° 2θ. In detail, the 2-phenyl-4-(3-carbomethoxypropionyl)-1,3-oxazolin-5-one purified according to the invention has the following XRPD powder diffractogram *):

| Peak number | 2⊖ (°) | Intensity (%) |
|---|---|---|
| 1 | 7.8 | 30.9 |
| 2 | 9.1 | 23.0 |
| 3 | 13.3 | 18.9 |
| 4 | 16.6 | 87.3 |
| 5 | 19.5 | 28.7 |
| 6 | 19.7 | 22.3 |
| 7 | 20.4 | 100.0 |
| 8 | 23.0 | 17.1 |
| 9 | 23.6 | 31.2 |
| 10 | 23.9 | 54.7 |
| 11 | 24.1 | 24.5 |
| 12 | 24.5 | 51.9 |
| 13 | 25.1 | 13.3 |
| 14 | 26.1 | 25.0 |
| 15 | 28.0 | 17.2 |

For comparison, the red-colored 2-phenyl-4-(3-carbomethoxypropionyl)-1,3-oxazolin-5-one of the crystalline form A is characterized in detail by the following XRPD powder diffractogram *):

| Peak number | 2⊖ (°) | Intensity (%) |
|---|---|---|
| 1 | 6.2 | 15.4 |
| 2 | 6.4 | 65.5 |
| 3 | 17.6 | 16.1 |
| 4 | 19.1 | 37.6 |
| 5 | 20.2 | 39.7 |
| 6 | 21.9 | 25.9 |
| 7 | 23.5 | 10.0 |
| 8 | 26.6 | 19.1 |
| 9 | 27.0 | 100.0 |
| 10 | 27.8 | 19.4 |

*) Note:
Only signals with an intensity of ≥10% are shown. The two XRPD powder diffractograms shown as a list were recorded under the same conditions, i.e., each with a STOE Powder Diffraction System using CuKα radiation (40 kV, 25 mA). The accuracy of the 2⊖ values was ±0.2°.

2-phenyl-4-(3-carboalkoxypropionyl)-1,3-oxazolin-5-one purified by the process according to the invention, and in particular in this case 2-phenyl-4-(3-carbomethoxypropionyl)-1,3-oxazolin-5-one, can be used to prepare 5-aminolevulinic acid hydrochloride or other salts of 5-aminolevulinic acid. For example, it can be converted into 5-aminolevulinic acid hydrochloride by hydrolysis in the presence of hydrochloric acid, for example according to the preparation procedure known from DE 22 08 800 (A1). In this case, it is advantageous that the preparation of 5-aminolevulinic acid hydrochloride or other salts of 5-aminolevulinic acid can be carried out with a purer 2-phenyl-4-(3-carboalkoxypropionyl)-1,3-oxazolin-5-one starting product in the form of the 2-phenyl-4-(3-carboalkoxypropionyl)-1,3-oxazolin-5-one purified according to the invention. In this respect, the present invention also relates to its use for the preparation of 5-aminolevulinic acid hydrochloride by hydrolysis in the presence of hydrochloric acid or for the preparation of other salts of 5-aminolevulinic acid.

Embodiment: The procedure was initially as in Example 1 from DE 22 08 800 (A1). For this purpose, 56 ml of succinic acid monomethyl ester chloride were added to a solution of 40 g of N-benzoylglycine in 200 ml of 4-methylpyridine, which had been placed in a three-necked flask of 500 ml capacity equipped with a thermometer, stirrer and dropping funnel and cooled with ice and salt, with stirring at such a rate that the temperature of the reaction compound was in the range of from −5 to 0° C.

After completion of the addition of acid chloride, the reaction compound was stirred for a further 5 hours at this temperature, then it was poured into a mixture of 1 kg of ice with 160 ml of concentrated hydrochloric acid while stirring.

The precipitated red-colored crystals of 2-phenyl-4-(3-carbomethoxypropionyl)-1,3-oxazolin-5-one (crystalline form A) were filtered off.

10 g of the red-colored crystals were washed with 45 ml of water, filtered and then washed twice with 20 mL of dry ethyl acetate each. The solid was then suspended in 20 mL of moist ethyl acetate (3 vol. % water). The mixture was then stirred for 10 hours until the red coloration of the crystals as well as the red coloration of the ethyl acetate phase had disappeared. The suspension was filtered and the filtered-off rod-shaped crystals of 2-phenyl-4-(3-carbomethoxypropionyl)-1,3-oxazolin-5-one (crystalline form B) were then washed once with 20 mL of dry ethyl acetate and dried.

The yield was 7.1 g based on the 10 g of red-colored crystals.

The purity was determined by means of HPLC, using the following parameters: column (250×4.6 mm, RP-18, 5 μm), injection 5 μL, flow 1 mL/min, column temperature 25° C., UV detection at 254 nm, mobile phase A=0.1% trifluoroacetic acid (TFA) in water, mobile phase B=0.1% TFA in acetonitrile, gradient from 90% A to 10% A within 15 min. The purity determined in this way was 97.2 HPLC area %.

Regarding the XRPD measurement, reference is made to what was mentioned above in the description.

Comparative example 1: The procedure was the same as in the embodiment, with the difference that no moist ethyl acetate was used. The red color had not disappeared.

Comparative example 2: The procedure was the same as in the embodiment, with the difference that stirring was only carried out for 1 hour. The red color was still clearly visible.

1. A process for purifying 2-phenyl-4-(3-carboalkoxypropionyl)-1,3-oxazolin-5-one, which is formed by reacting N-benzoylglycine with succinic acid monoalkyl ester chloride in 4-methylpyridine and obtainable in the form of red-colored crystals after neutralization with an excess of hydrochloric acid, characterized in that the red-colored crystals are suspended in an organic solvent or a mixture of a plurality of organic solvents until decolorization, the organic solvent or solvents being selected from the group consisting of organic solvents having an elution strength E⁰ in the range of from 0.40 to 0.55, and the organic solvent or solvents having a water content in the range of from 1 to 10 vol. %.

2. The process according to claim 1, wherein the succinic acid monoalkyl ester chloride is a succinic acid mono-C1-C4-alkyl ester chloride and the 2-phenyl-4-(3-carboalkoxypropionyl)-1,3-oxazolin-5-one is a 2-phenyl-4-(3-carbo-C1-C4-alkoxypropionyl)-1,3-oxazolin-5-one.

3. The process according to claim 2, wherein the succinic acid mono-C1-C4-alkyl ester chloride is succinic acid monomethyl ester chloride and the 2-phenyl-4-(3-carbo-C1-C4-alkoxypropionyl)-1,3-oxazolin-5-one is 2-phenyl-4-(3-carbomethoxypropionyl)-1,3-oxazolin-5-one.

4. The process according to claim 1, wherein the organic solvent or mixture of a plurality of organic solvents has a water content in the range of from 2 to 5 vol. %.

5. The process according to claim 1, wherein the water content of the organic solvent or mixture of a plurality of organic solvents is dimensioned such that the organic solvent or solvents do not form a two-phase system with the water.

6. The process according to claim 1, wherein the suspension takes place in the temperature range of from 10 to 40° C.

7. The process according to claim 1, wherein the suspension takes place in a ratio of 20 to 100 g of the red-colored crystals per liter of organic solvent.

8. The process according to claim 1, wherein the suspension is achieved by stirring and/or shaking.

9. The process according to claim 1, wherein the purified 2-phenyl-4-(3-carboalkoxypropionyl)-1,3-oxazolin-5-one is separated as crystals by means of a solid-liquid separation process after suspension has been completed.

10. The process according to claim 9, wherein the separated crystals are dried.

11. A purified 2-phenyl-4-(3-carboalkoxypropionyl)-1,3-oxazolin-5-one obtainable by a process of either claim 9.

12. The purified 2-phenyl-4-(3-carboalkoxypropionyl)-1,3-oxazolin-5-one according to claim 11, wherein the 2-phenyl-4-(3-carboalkoxypropionyl)-1,3-oxazolin-5-one is a 2-phenyl-4-(3-carbo-C1-C4-alkoxypropionyl)-1,3-oxazolin-5-one.

13. The purified 2-phenyl-4-(3-carbo-C1-C4-alkoxypropionyl)-1,3-oxazolin-5-one according to claim 12, wherein the 2-phenyl-4-(3-carbo-C1-C4-alkoxypropionyl)-1,3-oxazolin-5-one is 2-phenyl-4-(3-carbomethoxypropionyl)-1,3-oxazolin-5-one.

14. A 2-phenyl-4-(3-carbomethoxypropionyl)-1,3-oxazolin-5-one in crystalline form B with a powder X-ray diffraction pattern comprising the following reflections: 7.8, 9.1, 13.3, 16.6, 19.5, 19.7, 20.4, 23.0, 23.6, 23.9, 24.1, 24.5, 25.1, 26.1 and 28.0±0.2° 2θ.

15. A use of a 2-phenyl-4-(3-carboalkoxypropionyl)-1,3-oxazolin-5-one according to claim 11 for the preparation of 5-aminolevulinic acid hydrochloride by hydrolysis in the presence of hydrochloric acid or for the preparation of other salts of 5-aminolevulinic acid.

16. A purified 2-phenyl-4-(3-carboalkoxypropionyl)-1,3-oxazolin-5-one obtainable by a process of claim 10.
